(19) United States
(12) Patent Application Publication
(10) Pub. No.: US 2025/0011761 A1
(43) Pub. Date: Jan. 9, 2025

(54) DIFFERENTIAL METHYLATION ENRICHMENT METHODS AND USES THEREOF

(71) Applicant: ARC BIO, LLC, Cambridge, MA (US)

(72) Inventor: Stephane B. GOURGUECHON, San Mateo, CA (US)

(21) Appl. No.: 18/711,823

(22) PCT Filed: Nov. 18, 2022
(86) PCT No.: PCT/US2022/080161

§ 371 Date: May 20, 2024

Related U.S. Application Data
(60) Provisional application No. 63/281,146, filed on Nov. 19, 2021.

(51) Int. Cl. C12N 15/10

(52) U.S. Cl. CPC C12N 15/1093

(57) ABSTRACT

The present invention provides methods for identifying genomic regions that are differentially CpG methylated in two samples. Also provided are novel methods for generating a DNA library that is enriched for or depleted of CpG-methylated DNA and enzyme compositions for use in the disclosed methods.

### CROSS-REFERENCE TO RELATED APPLICATIONS

This application claims priority to U.S. Provisional Application No. 63/281,146 filed on Nov. 19, 2021, the contents of which are incorporated by reference in their entireties.

### BACKGROUND

A differentially methylated genomic region is a genomic region that comprises different DNA methylation patterns in different samples. For example, a genomic region can be differentially methylated across samples from different cell types, tissues, subjects, or organisms. Differentially methylated regions are associated with different gene expression levels, and abnormal DNA methylation has been implicated in the development of various diseases, including cancer. Further, the genomes of some organisms comprise substantially higher levels of methylation than the genomes of other organisms. Thus, methylation status can be used to distinguish between DNA molecules from different cell-types (e.g., cancer cells vs. healthy cells) and different organisms (e.g., humans vs. bacteria). Methods that use methylation status to enrich for target DNA molecules in a sample would be useful for reducing sequencing costs and increasing depth of coverage.

### SUMMARY

In a first aspect, the present invention provides methods of identifying genomic regions that are differentially methylated in two samples. The methods comprise (a) providing two samples comprising DNA; (b) contacting the samples with one or more CpG methylation-sensitive restriction enzyme to generate cut sites or nick sites in the DNA at enzyme recognition sites; (c) ligating adapters to the cut sites or nick sites to generate DNA libraries; (d) sequencing the DNA libraries to generate sequencing reads; (e) mapping the sequencing reads to a reference genome; and (f) comparing the mapped sequencing reads from each sample to identify genomic regions that are differentially methylated in the two samples.

In a second aspect, the present invention provides compositions comprising at least one CpG methylation-sensitive restriction enzyme selected from the group consisting of AluI, DdeI, HpyCH4IV, HpaII, HaeIII, RsaI, and Sau3AI (i.e., the DRASH enzymes).

In a third aspect, the present invention provides methods of generating DNA libraries that are depleted of or enriched for CpG-methylated DNA.

### BRIEF DESCRIPTION OF THE DRAWINGS

FIG. 1 is a diagram illustrating an exemplary method for depleting CpG-methylated DNA from a sample using the DRASH enzymes. (A) DNA is terminally dephosphorylated using the enzyme recombinant shrimp alkaline phosphatase (rSAP) and treated with the CpG methylation-sensitive restriction enzymes AluI, DdeI, HpyCH4IV, HpaII, HaeIII, RsaI, and Sau3AI (i.e., the DRASH enzymes), whose activity is blocked by the presence of CpG methylation. The DRASH enzymes generate cut sites with exposed terminal phosphates at enzyme recognition sites that lack CpG methylation. (B) Adapters are ligated to the DNA to generate DNA libraries. (C) 5′ phosphorylation is needed for a DNA molecule to be used as a substrate for DNA ligase. Thus, only the unmethylated sequences that were cut by the DRASH enzymes will comprise adapters on both ends, allowing them to be selectively amplified via PCR.

FIG. 2 is a diagram illustrating an exemplary method for identifying genomic regions that are differentially methylated in two samples using the DRASH enzymes. (A) The DNA in two samples (e.g., from two different populations of human cells) is terminally dephosphorylated using the enzyme rSAP and treated with the CpG methylation-sensitive DRASH enzymes. The DRASH enzymes generate cut sites with exposed terminal phosphates at enzyme recognition sites that lack CpG methylation. (B) Adapters are ligated to the DNA to generate DNA libraries that are enriched for the unmethylated sequences that were cut by the DRASH enzymes. (C) The DNA libraries are sequenced, and the resulting sequencing reads are mapped to a reference genome. The mapped sequencing reads from each sample are then compared to identify genomic regions that are differentially methylated in the two samples.

FIG. 3 is a diagram illustrating an exemplary method for identifying genomic regions that are differentially methylated in two samples using the nickase Nt.CviPII. (A) The DNA in two samples is terminally dephosphorylated using the enzyme rSAP and treated with the CpG methylation-sensitive nickase Nt.CviPII, whose activity is blocked by the presence of CpG methylation. Nt.CviPII generates nick sites with exposed terminal phosphates at enzyme recognition sites that lack CpG methylation. (B) Adapters are ligated to the DNA to generate single-stranded DNA libraries that are enriched for the unmethylated sequences that were cut by Nt.CviPII. (C) The DNA libraries are sequenced, and the resulting sequencing reads are mapped to a reference genome. The mapped sequencing reads from each sample are then compared to identify genomic regions that are differentially methylated in the two samples.

FIG. 4 is a diagram illustrating an exemplary method for identifying genomic regions that are differentially methylated in two samples using the enzymes FspEI and MspJI. (A) The DNA in two samples is terminally dephosphorylated using the enzyme rSAP and treated with the CpG methylation-sensitive restriction enzymes FspEI and MspJI, which are only active in the presence of CpG methylation. FspEI and MspJI generate cut sites with exposed terminal phosphates at enzyme recognition sites that comprise CpG methylation. (B) Adapters are ligated to the DNA to generate DNA libraries that are enriched for the methylated sequences that were cut by Nt.CviPII. (C) The DNA libraries are sequenced, and the resulting sequencing reads are mapped to a reference genome. The mapped sequencing reads from each sample are then compared to identify genomic regions that are differentially methylated in the two samples.

FIG. 5 provides boxplot graphs showing the sequence read quantification score for portions of test samples spiked with four quantities (i.e., 0 copies/mL, 20-40 copies/mL, 100-200 copies/mL, or 500-1000 copies/mL) of the test organisms Bordetella pertussis, Staphylococcus aureus, Escherichia coli, and Streptococcus agalactiae and fragmented using either the fragmentase enzymes (Frag) or the DRASH enzymes (DRASH).

### DETAILED DESCRIPTION

The present invention provides methods for identifying genomic regions that are differentially CpG methylated in two samples. Also provided are novel methods for generating a DNA library that is enriched for or depleted of CpG-methylated DNA and enzyme compositions for use in the disclosed methods.

“CpG methylation” is DNA methylation that occurs at a CpG site. A “CpG site” is a region of DNA wherein the nucleotide cytosine is followed by the nucleotide guanine in the 5′ to 3′ direction. In DNA, adjacent nucleotides are linked by a phosphodiester bond, i.e., a covalent bond formed between the 5′ phosphate group of one nucleotide and the 3′-OH group of another. The “p” in “CpG” site represents the 5′ phosphate group. In CpG methylation, the cytosine in the CpG dinucleotide is methylated to form 5-methylcytosine via addition of a methyl group by a DNA methyltransferase.

CpG methylation occurs more frequently in the genomes of vertebrates as compared to those of bacteria, fungi, and viruses. For example, mammals have substantial CpG methylation whereas fungi have low levels (e.g., 0.1-0.5%) and bacteria only have methylation at specific genomic regions. Thus, CpG methylation status can be used to distinguish between the DNA of a mammalian host and a pathogen.

CpG methylation plays a critical role in regulating gene expression. For example, genes are stably silenced by the presence of multiple methylated CpG sites within their promoters. In cancers, gene silencing is driven by promoter hypermethylation about 10 times more frequently than it is by DNA mutations. Thus, CpG methylation status can be used as an indicator of gene activity or to distinguish between diseased and healthy states.

In the methods of the present invention, samples are depleted of or enriched for CpG-methylated DNA. CpG methylation-sensitive restriction enzymes are used to cleave DNA at recognition sites that are either (1) CpG methylated or (2) not CpG methylated. Cleavage of the DNA by these enzymes generates DNA fragments with exposed terminal phosphate groups to which adapters can be ligated. Ligating adapters to only the cleaved DNA fragments allows one to selectively isolate, amplify, and/or sequence genomic regions that contain or lack CpG methylation.

### Methods of Identifying Differential Methylation:

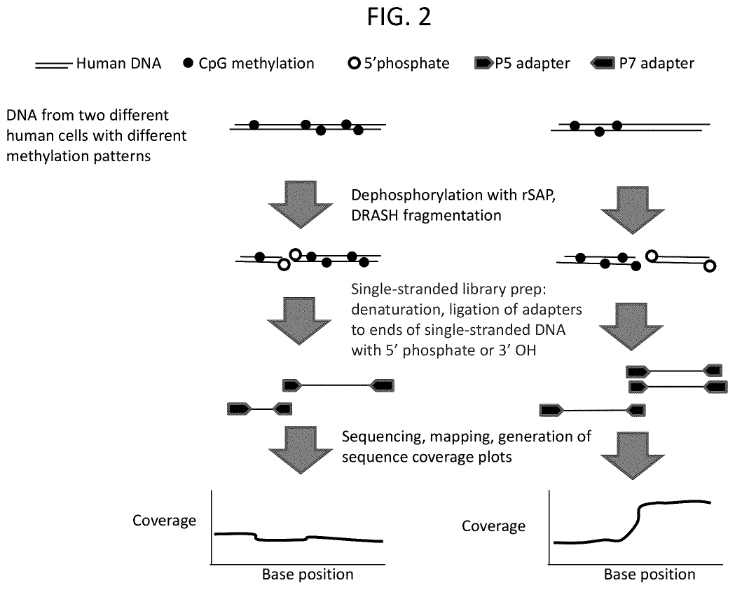

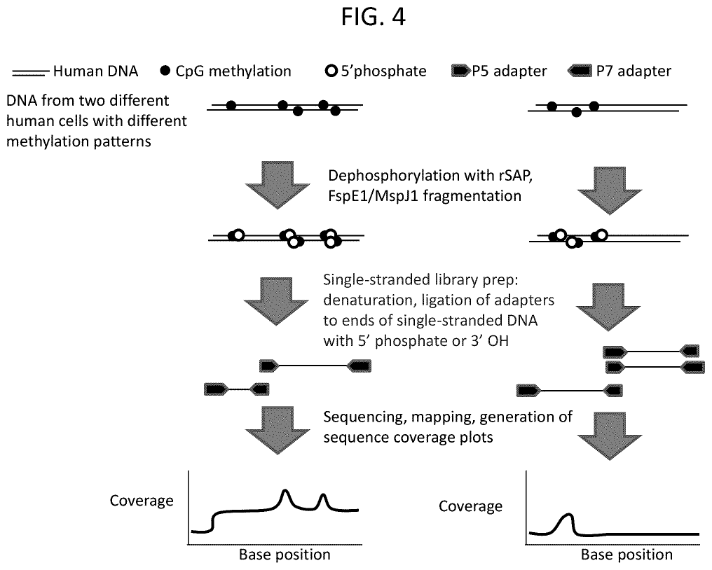

In a first aspect, the present invention provides methods of identifying genomic regions that are differentially methylated in two samples (see FIGS. 2-4). The methods comprise (a) providing two samples comprising DNA; (b) contacting the samples with one or more CpG methylation-sensitive restriction enzyme to generate cut sites or nick sites in the DNA at enzyme recognition sites; (c) ligating adapters to the cut sites or nick sites to generate DNA libraries; (d) sequencing the DNA libraries to generate sequencing reads; (e) mapping the sequencing reads to a reference genome; and (f) comparing the mapped sequencing reads from each sample to identify genomic regions that are differentially methylated in the two samples.

A genomic region is “differentially methylated” between two samples if it is methylated in one sample and not the other. For example, a genomic region may be differentially methylated between two samples from different cell types, tissues, subjects, or organisms. A differentially methylated genomic region may be as small as a single CpG site or may span many kilobases of the genome. The methods of the present invention can be used to identify differentially methylated regions that are hundreds of bases in length as well as those that are much smaller.

Any sample comprising DNA may be used in the various methods of the present invention. Suitable samples include, without limitation, biological samples, clinical samples, forensic samples, and environmental samples. Exemplary clinical and forensic samples include, but are not limited to, whole blood, plasma, serum, tears, saliva, mucous, cerebrospinal fluid, teeth, bone, fingernails, feces, urine tissue, and biopsy samples. In some embodiments, the DNA in the sample is fragmented. In some embodiments, the DNA molecules in the sample are about 20 to about 5000 base pairs (bp) in length, about 20 to about 1000 bp in length, about 20 to about 500 bp in length, about 20 to about 400 bp in length, about 20 to about 300 bp in length, about 20 to about 200 bp in length, about 20 to 100 bp in length, about 50 to about 5000 bp in length, about 50 to about 1000 bp in length, about 50 to about 500 bp in length, about 50 to about 400 bp in length, about 50 to about 300 bp in length, about 50 to about 200 bp in length, about 50 to about 100 bp in length, about 100 to about 5000 bp in length, about 100 to about 1000 bp in length, about 100 to about 500 bp in length, about 100 to about 400 bp in length, about 100 to about 300 bp in length, or about 100 to about 200 bp in length.

In the disclosed methods, samples are contacted with one or more CpG methylation-sensitive restriction enzyme to generate cut sites or nick sites in the DNA at enzyme recognition sites. As used herein, the term “CpG methylation-sensitive restriction enzyme” refers to a restriction enzyme that is sensitive to the presence of CpG methylation within its cognate recognition site or adjacent to its cognate recognition site (e.g., within 1-50 nucleotides). The term “enzyme recognition site” or “recognition site”, as used herein, refers to a specific DNA sequence that is recognized by a restriction enzyme. Some restriction enzymes cut within their recognition sites, while others cut adjacent to their recognition sites (e.g., within 1-105 nucleotides of the recognition site). In some embodiments, the recognition site is between 3-20 bp in length. However, in preferred embodiments, the recognition site is relatively short (e.g., 3-5 bp in length), such that the CpG methylation-sensitive restriction enzyme cleaves the DNA with greater frequency.

In the present methods, the CpG methylation-sensitive restriction enzyme(s) are used to generate cuts or nicks at their cognate recognition sites. As used herein, the term “cutting” refers to a reaction that breaks the phosphodiester bonds between two adjacent nucleotides in both strands of a double-stranded DNA molecule, resulting in a double-stranded break, and the term “cut site” refers to a site at which a DNA molecule has been cut. In contrast, the term “nicking” refers to a reaction that breaks the phosphodiester bond between two adjacent nucleotides in only one strand of a double-stranded DNA molecule, resulting in a single-stranded break, and the term “nick site” refers to a site at which a DNA molecule has been nicked. The term “cleaving” is used herein to refer generally to a reaction in which DNA is either cut or nicked.

In some embodiments, the one or more CpG methylation-sensitive restriction enzyme comprises a mixture of at least two, at least three, at least four, at least five, at least six, at least seven, at least eight, at least nine, or at least ten different CpG methylation-sensitive restriction enzymes.

In some embodiments, the activity of the one or more CpG methylation-sensitive restriction enzyme is blocked by CpG methylation within or adjacent to its cognate recognition site. Such enzymes cleave DNA at recognition sites that lack CpG methylation, and do not cleave or cleave at reduced levels at recognition sites that contain CpG methylation. Suitable CpG methylation-sensitive restriction enzymes that cannot cleave at genomic sites that are CpG methylated include, without limitation, AatII, AccII, AluI, Aor13HI, Aor51HI, BspT104I, BssHII, Cfr10I, ClaI, CpoI, DdeI, Eco52I, HaeII, HapII, HhaI, HpyCH4IV, MluI, NaeI, NotI, NruI, NsbI, Nt.CviPII, PmaCI, Psp1406I, PvuI, RsaI, SacII, SalI, SmaI, SnaBI, and Sau3AI.

For use with this present invention, the inventors have developed mixtures of seven CpG methylation-sensitive restriction enzymes that are blocked by CpG methylation, i.e., AluI, DdeI, HpyCH4IV, HpaII, HaeIII, RsaI, and Sau3AI, which are referred to herein as “the DRASH enzymes”. Thus, in some embodiments (depicted in FIG. 2), the one or more CpG methylation-sensitive restriction enzyme comprises at least one of the seven DRASH enzymes. Because each of the DRASH enzymes has a different recognition sequence at which it cleaves unmethylated DNA, the use of multiple DRASH enzymes results in more frequent cleavage and greater genomic coverage. Thus, in some embodiments, the one or more CpG methylation-sensitive restriction enzyme comprises at least two, at least three, at least four, at least five, at least six, or all seven of the DRASH enzymes.

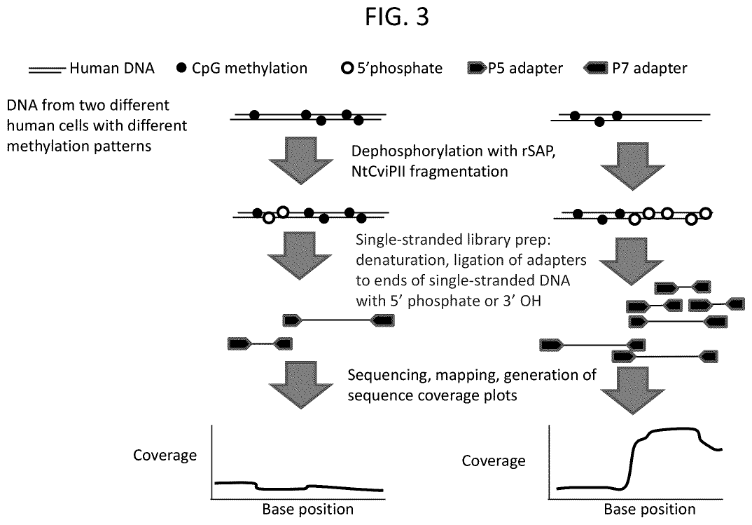

In some embodiments (depicted in FIG. 3), the one or more CpG methylation-sensitive restriction enzyme comprises Nt.CviPII. NtCviPII is a nickase that has a DNA recognition site that is relatively short (i.e., three bases). As a result, this enzyme nicks more frequently throughout the genome than other enzymes with longer recognition sites.

In other embodiments, the activity of the one or more CpG methylation-sensitive restriction enzyme requires CpG methylation within or adjacent to its cognate recognition site. Such enzymes cleave DNA at recognition sites that contain CpG methylation, and do not cleave or cleave at reduced levels at recognition sites that lack CpG methylation. Suitable CpG methylation-sensitive restriction enzymes that require CpG methylation include, without limitation, AbaSI, FspEI, LpnPI, MspJI, and McrBC. In particular embodiments, the one or more CpG methylation-sensitive restriction enzyme comprises FspEI and MspJI.

Cleavage of DNA by a CpG methylation-sensitive restriction enzyme generates DNA fragments with an exposed terminal (5′) phosphate, which is required for ligation. As a result, in step (c) of the present methods, adapters are ligated to the cut/nick sites but not to uncut/unnicked sites. As used herein, the term “ligating” refers to a reaction in which DNA ligase joins two DNA molecules via the formation of two covalent phosphodiester bonds between the 3′ hydroxyl group of one DNA molecule and the 5′ phosphate group of the other DNA molecule in an ATP-dependent reaction.

As used herein, an “adapter” is a DNA sequence that is added to a DNA molecule to facilitate its amplification, isolation, or sequencing. Adapters may be double-stranded or single-stranded. The structure of an adapter may be linear, Y-shaped, circular, or hairpin-shaped. The ligatable end of the adapter may be designed to be compatible with the overhangs or blunt ends generated via cleavage by a CpG methylation-sensitive restriction enzyme. In some embodiments, the adapters are 10 to 100 bp in length. In specific embodiments, the adapters are at least 10 bp, at least 15 bp, at least 20 bp, at least 25 bp, at least 30 bp, at least 35 bp, at least 40 bp, at least 45 bp, at least 50 bp, at least 55 bp, at least 60 bp, at least 65 bp, at least 70 bp, at least 75 bp, at least 80 bp, at least 85 bp, at least 90 bp, or at least 95 bp in length.

In some embodiments, addition of the adapter sequences adds primer binding sites to the DNA molecules such that the DNA that was cleaved by the CpG methylation-sensitive restriction enzyme(s) can be selectively amplified using a PCR-based method.

In other embodiments, the adapter sequence binds to a particular capture molecule enabling isolation of the DNA that was cleaved by the CpG methylation-sensitive restriction enzyme(s). For example, an adapter can hybridize with a capture molecule comprising a complementary DNA sequence, or an adapter may include a tag (e.g., biotin) that binds to a particular capture molecule (e.g., streptavidin). Suitable tags include, without limitation, 6-Histidine (His), hemagglutinin (HA), cMyc, GST, Flag, V5, and NE tags.

In preferred embodiments, the adapters are sequencing adapters, i.e., sequences that are designed to interact with a specific sequencing platform (e.g., the surface of an Illumina flow cell) to facilitate a sequencing reaction. The optimal length of a sequencing adapter will vary depending on the sequencing platform used. One of ordinary skill will understand that adapter sequences may be as short as 20 nucleotides or substantially longer. For example, an adapter sequence of 58 nucleotides may be used with an Illumina machine. In some embodiments, the sequencing adapters comprise unique molecular identifier (UMI) sequences, which comprise a sequence label (e.g., a random DNA sequence) that is unique to each DNA molecule to enable its quantification. In some embodiments, the UMI comprises 2, 3, 4, 5, 6, 7, 8, 9, 10, 11, 12, or more nucleotides. In some embodiments, the sequencing adapters comprise “barcode” sequences, which are used to label all DNA molecules from a particular sample or source (e.g., DNA from a particular cell-type, tissue, subject, or organism). The inclusion of barcodes in the adapters allows multiple sequencing libraries to sequenced simultaneously during a single run, thereby reducing sequencing costs. A barcode sequence may be 4, 5, 6, 7, 8, 9, 10, 11, 12, 13, 14, 15, 16, or more nucleotides in length. A barcode sequence may be included at the 5′-end, the 3′-end, or in the middle of a DNA molecule.

As used herein, a “DNA library” is a collection of DNA fragments to which adapters have been ligated to enable downstream applications. Any method for preparing a DNA library may be used with the present invention. Most DNA library preparation methods produce libraries that comprise double-stranded DNA. However, there are several methods that can be used to produce libraries comprising single-stranded DNA. One such method is the single reaction single-stranded library (SRSLY) method. For a detailed description of SRSLY, see BMC Genomics (2019) 20(1):1023, which is incorporated by reference in its entirety. SRSLY can be used to prepare a single-stranded DNA library as part of any of the methods disclosed herein.

Any sequencing method may be used with the present invention. Suitable methods include, for example, Sanger sequencing, Illumina sequencing, single molecule real time (SMRT) sequencing, Nanopore DNA sequencing, massively parallel signature sequencing (MPSS), Polony sequencing, 454 pyrosequencing, combinatorial probe anchor synthesis (cPAS), Ion Torrent semiconductor sequencing, DNA nanoball sequencing, and SOLiD sequencing. For high-throughput applications, the sequencing method is advantageously a next-generation sequencing method.

DNA sequencing produces “sequencing reads,” i.e., inferred nucleotide sequences that correspond to all or part of a single DNA fragment. In the present methods, sequencing reads are mapped to (i.e., assigned to a specific location with) a reference genome to allow for comparison of methylation patterns between two samples. A “reference genome” is a digital DNA sequence database that is used as a representative example of a genome of one idealized individual organism. Many bioinformatic tools that allow one to map sequencing reads and to compare mapped sequencing reads between samples are available, including many that are available freely online (e.g., Galaxy). Using methods of the present invention, differential methylation can be identified as a difference in the read coverage or depth at a particular genomic base position (i.e., there is a greater number of sequencing reads that map to a particular base position in one sample than in the other), as depicted in part (C) of FIGS. 2-4.

In the present methods, adapters are selectively ligated to sites that were cut/nicked by a CpG methylation-sensitive restriction enzyme because these sites contain a 5′ phosphate. Thus, it may be advantageous to remove 5′ phosphates that are present on the ends of the DNA at the onset of the methods such that adapters are not ligated to DNA molecules with preexisting phosphates, allowing them to contribute to background noise. Accordingly, in some embodiments, the methods further comprise terminally dephosphorylating the DNA prior to step (b). As used herein, the term “terminally dephosphorylated” refers to DNA molecules that have had the phosphate group removed from their 5′ end.

Dephosphorylation can be accomplished using any phosphatase. Phosphatases are enzymes that catalyze dephosphorylation reactions. Exemplary phosphatases include, but are not limited to, shrimp alkaline phosphatase (SAP), recombinant shrimp alkaline phosphatase (rSAP), calf intestine alkaline phosphatase (CIP), and Antarctic phosphatase.

### Compositions:

In a second aspect, the present invention provides compositions comprising at least one CpG methylation-sensitive restriction enzyme selected from the group consisting of AluI, DdeI, HpyCH4IV, HpaII, HaeIII, RsaI, and Sau3AI (i.e., the DRASH enzymes). In preferred embodiments, the composition comprises at least four of the DRASH enzymes. In some embodiments, the composition comprises at least five of the DRASH enzymes. In some embodiments, the composition comprises AluI, DdeI, HpyCH4IV, RsaI, and Sau3AI. In some embodiments, the composition comprises at least six of the DRASH enzymes. In some embodiments, the composition comprises all seven DRASH enzymes. These compositions are referred to herein as “DRASH enzyme compositions.”

Methods of Enriching for/Depleting Methylated DNA:

In a third aspect, the present invention provides methods of generating DNA libraries that are depleted of or enriched for CpG-methylated DNA. The DNA libraries produced by these methods have reduced complexity and can be used in a variety of downstream applications including, but not limited to, PCR amplification, cloning, high throughput sequencing, identification of rare sequences, and quantification of sequences within a library.

In some embodiments, the methods generate a DNA library that is depleted of CpG-methylated DNA. In other embodiments, the methods generate a DNA library that is enriched for CpG-methylated DNA, i.e., by selectively depleting unmethylated sequences. The DNA library may be depleted of or enriched for unwanted CpG-methylated DNA by at least about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 25 fold, about 30 fold, about 40 fold, about 50 fold, about 100 fold, about 200 fold, about 500 fold, or about 1000 fold. In some embodiments, the sample is depleted of or enriched for CpG-methylated DNA by at least about 50% to about 70%. In some embodiments, the sample is depleted of or enriched for CpG-methylated DNA by at least about 95%.

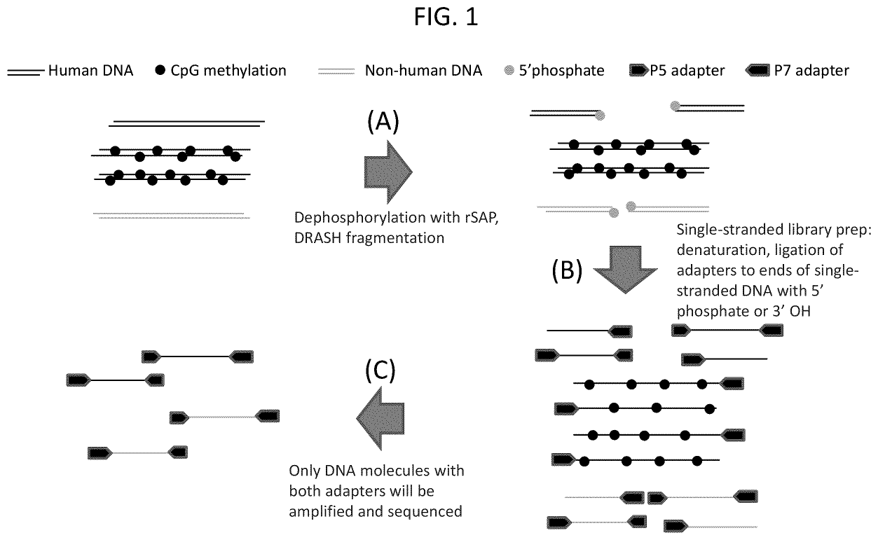

In a first embodiment, depicted in FIG. 1, the methods involve generating a DNA library that is depleted of CpG-methylated DNA using one or more DRASH enzymes. These methods comprise (a) providing a sample comprising DNA; (b) contacting the sample with a DRASH enzyme composition described herein to generate cut sites in the DNA at DRASH enzyme recognition sites that lack CpG methylation; and (c) ligating adapters to the cut sites to generate a DNA library that is depleted of CpG-methylated DNA.

In a second embodiment, the methods involve generating a single-stranded DNA library that is depleted of CpG-methylated DNA. The methods comprise: (a) providing a sample comprising DNA; (b) contacting the sample with NtCviPII to generate nick sites in the DNA at NtCviPII recognition sites that lack CpG methylation; and (c) ligating adapters to the nick sites to generate a single-stranded DNA library that is depleted of CpG-methylated DNA. As is discussed above, use of the nickase NtCviPII yields more precise mapping because it cuts more frequently in the genome than other enzymes due to its short (3 base pair) recognition site. A nickase can be used in methods in which the resulting DNA libraries are single stranded because the nicks it generates will become breaks as the DNA strands are separated.

In a third embodiment, the methods involve generating a DNA library that is enriched for CpG-methylated DNA. The methods comprise: (a) providing a sample comprising DNA; (b) contacting the sample with FspE1 and/or MspJ1 to generate cut sites in the DNA at FspE1 and/or MspJ1 recognition sites comprising CpG methylation; and (c) ligating adapters to the cut sites to generate a DNA library that is enriched for CpG-methylated DNA. Because these methods enrich for the set of genomic regions that are depleted by the depletion methods described above, they can be used to confirm the results of the depletion methods.

In the present methods, adapters are selectively ligated to sites that were cut/nicked by a CpG methylation-sensitive restriction enzyme because they contain a 5′ phosphate. Thus, it may be advantageous to remove 5′ phosphates that are present on the ends of the DNA at the onset of the methods such that adapters are not ligated to DNA molecules with preexisting phosphates, allowing them to contribute to background noise. Accordingly, in some embodiments, the methods further comprising terminally dephosphorylating the DNA prior to step (b). Dephosphorylation can be accomplished using any phosphatase, as described above.

The present methods can be used to enrich for either (1) DNA that comprises high levels of CpG methylation or (2) DNA that lacks CpG methylation or has low levels of CpG methylation.

The DNA of mammals contains substantially higher levels of CpG methylation than the DNA of pathogens. Thus, by enriching for or depleting a sample of CpG-methylated DNA, the present methods can be used to distinguish between mammalian DNA and the DNA of a pathogenic organism. For example, the methods can be used to enrich for either (1) the DNA of a mammalian host organism or (2) the DNA of a pathogenic organism that is present within the mammalian host. Thus, in some embodiments, the sample comprises both DNA from a mammalian organism and DNA from a pathogenic organism. Suitable mammalian organisms include, without limitation, humans, horses, sheep, cows, pigs, donkeys, cats, dogs, gerbils, mice, rats, and monkeys. In some embodiments, the mammalian organism is a human. Suitable pathogenic organisms include bacteria, yeast, viruses, and parasites.

In mammals, CpG methylation occurs more frequently in the genome at transcriptionally active sites than at transcriptionally silent sites. Thus, CpG methylation can be used to enrich for active or inactive regions of a mammalian genome. Examples of transcriptionally active regions include promoters and transcriptionally active genes.

In some embodiments, the methods further comprise an additional depletion and/or enrichment step. In some embodiments, the disclosed methods are combined with nucleic acid-guided nuclease-based depletion methods. For example, in some embodiments, the methods further comprise contacting the sample, after step (c), with a nucleic acid-guided nuclease and guide nucleic acids (gNAs), wherein the gNAs are complementary to sites within DNA molecules that are targeted for depletion, thereby generating cut DNA molecules that are adapter-ligated on only one end. Nucleic acid-guided nuclease-based enrichment methods are described in WO/2016/100955, WO/2017/031360, WO/2017/100343, WO/2017/147345, and WO/2018/227025, the contents of which are incorporated by reference in their entirety.

As used herein, a “nucleic acid-guided nuclease” is a nuclease that cleaves DNA, RNA or DNA/RNA hybrids, and that uses one or more guide nucleic acids (gNAs) to confer specificity. A nucleic acid-guided nuclease can be a DNA-guided DNA nuclease, a DNA-guided RNA nuclease, an RNA-guided DNA nuclease, or an RNA-guided RNA nuclease. A nucleic acid-guided nuclease can be an endonuclease or an exonuclease. A nucleic acid-guided nuclease may be naturally occurring or engineered. In some embodiments, the nucleic acid-guided nuclease is selected from the group consisting of Cas9, Cpf1, Cas3, Cas8a-c, Cas10, Cas13, Cas14, Cse1, Csy1, Csn2, Cas4, Csm2, Cm5, Csf1, C2c2, CasX, CasY, Cas14, and NgAgo. The nucleic acid-guided nuclease can be from any bacterial or archaeal species. For example, in some embodiments, the nucleic acid-guided nuclease is from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis Corynebacter diphtheria, Acidaminococcus, Lachnospiraceae bacterium, or Prevotella.

A “guide nucleic acid (gNA)” is a nucleic acid that targets a nucleic acid-guided nuclease to a specific genomic sequence via complementary base pairing. The gNAs used with the present invention comprise a sequence that is complementary to a portion of a DNA molecule that is targeted for depletion (i.e., the target sequence). The complementary portion of a gNA comprises at least 10 contiguous nucleotides, and often comprises 17-23 contiguous nucleotides that are complementary to the target sequence. The complementary portion of the gNA may be partially or wholly complementary to the target sequence. In some embodiments, the gNA is from 20 to 120 bases in length, or more. In certain embodiments, the gNA can be from 20 to 60 bases, 20 to 50 bases, 30 to 50 bases, or 39 to 46 bases in length. Various online tools and software environments can be used to design an appropriate gNA for a particular application. The gNA may comprise DNA and/or RNA. In some embodiments, the gNA is a chemically modified gNA. For example, the gNA may be chemically modified to decrease a cell's ability to degrade the gNA. Suitable chemically modified gNAs may include one or more of the following modifications: 2′-fluoro (2′-F), 2′-O-methyl (2′-OMe), S-constrained ethyl (cEt), 2′-O-methyl (M), 2′-O-methyl-3′-phosphorothioate (MS), and/or 2′-O-methyl-3′-thiophosphonoacetate (MSP). In some embodiments, the gNA is composed of two molecules that base pair to form a functional gRNA: one comprising the region that binds to the nucleic acid-guided nuclease and one comprising a targeting sequence that binds to the target site. Alternatively, the gNA may be a single molecule comprising both of these components, e.g., a single guide RNA (sgRNA).

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### EXAMPLES

### Example 1: Library Preparation with Differential Enrichment Based on Methylation Status

Test samples were prepared with no spike in or low (20-40 copies/mL), medium (100-200 copies/mL), or high (500-1000 copies/mL) titer level spike-ins of Bordetella pertussis, Escherichia coli, Epstein-Barr virus (EBV), adenovirus C (ADV-C), BK virus (BKV), John Cunningham virus (JCV), human herpesvirus 6A (HHV6A), human herpesvirus 6B (HHV6B), Staphylococcus aureus, Streptococcus agalactiae, parvovirus B19 (B19), and varicella-zoster virus (VZV). The test samples were prepared for sequencing using standard methods (i.e., the single reaction single-stranded library (SRSLY) method), except that a first portion of each sample was fragmented using fragmentase (i.e., NEBNext® dsDNA Fragmentase®) while a second portion of each sample was fragmented using the DRASH enzymes AluI, DdeI, HpyCH4IV, RsaI, and Sau3AI. Fragmentase contains two enzymes: one that randomly nicks double-stranded DNA and another cuts the strand opposite to the nicks. Thus, fragmentase generates random fragmentation similar to that generated using mechanical methods.

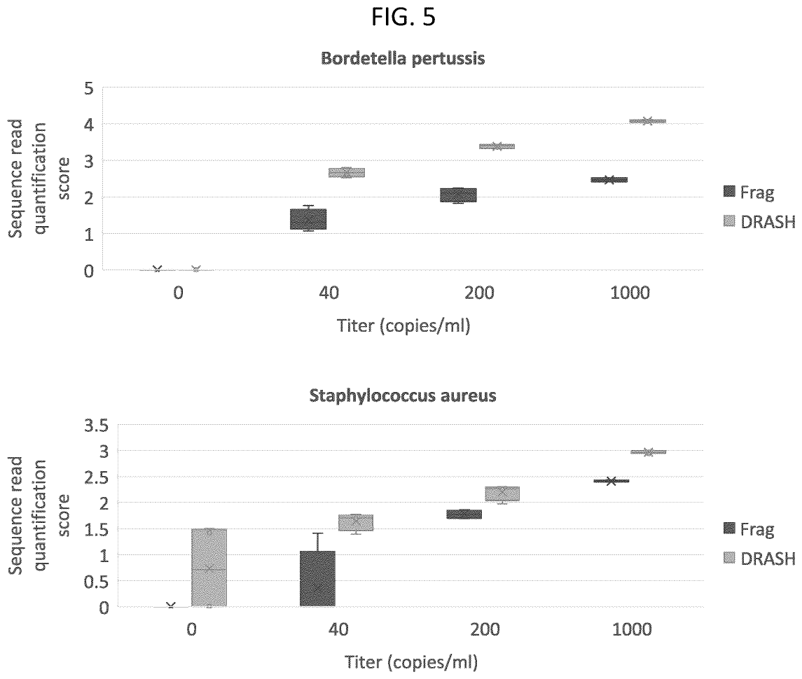

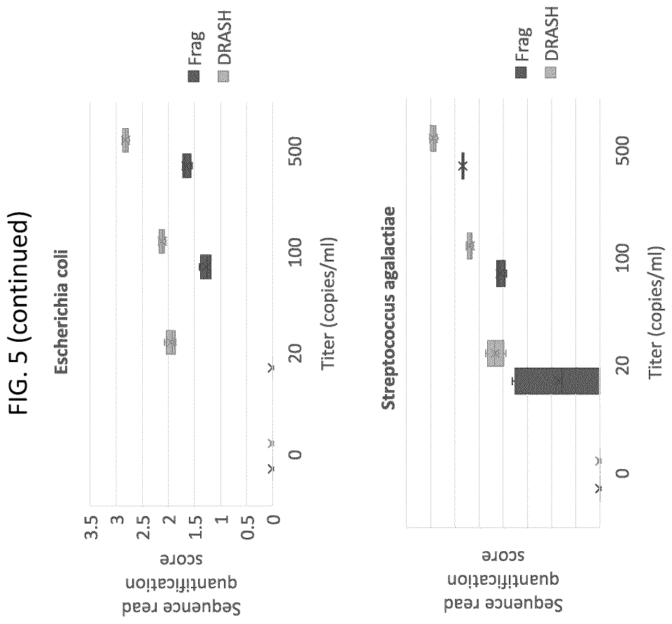

Table 1 shows the sequence read quantification score for the portions of the samples that were fragmented with the fragmentase enzymes, while Table 2 shows the sequence read quantification score for the portions of the samples that were fragmented with the DRASH enzymes. FIG. 5 provides graphs of these results for the B. pertussis, S. aureus, E. coli, and S. agalactiae test samples.

For several organisms, including E. coli, JCV, HHV6A, HHV6B, S. aureus, and S. agalacticae, a greater number of sequencing reads were produced via fragmentation with the DRASH enzymes as compared to the fragmentase enzymes. However, for a few organisms, including ADV-C, B19, and VZV, fewer sequencing reads were produced via fragmentation with the DRASH enzymes as compared to the fragmentase enzymes. These results demonstrate that fragmentation with the DRASH enzymes can be used to enrich for or deplete the DNA of certain organisms from a sequencing library.

TABLE 1
Sequence read quantification score for
fragmentase enzyme-fragmented samples

| | | | | |
|---|---|---|---|---|
| | | Low spike- | Medium spike- | High spike- |
| | No | in (20-40 | in (100-200 | in (500-1k |
| Organism | spike-in | copies/mL) | copies/mL) | copies/mL) |

| | | | |
|---|---|---|---|
| B. pertussis | 1.36 | 2.06 | 2.46 |
| E. coli | | 1.27 | 1.65 |
| EBV | | 1.16 | 0.98 |
| ADV-C | | | 2.08 |
| BKV | | | 2.17 |
| JCV | | | 2.9 |
| HHV6A | 0.81 | 1.21 | 1.39 |
| HHV6B | 0.8 | 1.23 | 1.38 |
| S. aureus | 1.41 | 1.77 | 2.41 |
| S. agalactiae | 1.68 | 2.05 | 2.83 |
| B19 | | | 2.92 |
| VZV | 2.03 | 1.31 | 2.13 |

TABLE 2
Sequence read quantification score
for DRASH enzyme-fragmented samples

| | | Low spike- | Medium spike- | High spike- |
|---|---|---|---|---|
| | No | in (20-40 | in (100-200 | in (500-1k |
| Organism | spike-in | copies/mL) | copies/mL) | copies/mL) |
| B. pertussis | | 2.65 | 3.38 | 4.07 |
| E. coli | | 1.94 | 2.15 | 2.81 |
| EBV | 2.45 | | 2.24 | 2.65 |
| ADV-C | | | | 2.62 |
| BKV | | 3.23 | | 3.3 |
| JCV | | 2.74 | | 3.41 |
| HHV6A | | 1.6 | 1.84 | 2.4 |
| HHV6B | | 1.59 | 1.83 | 2.41 |
| S. aureus | 1.5 | 1.63 | 2.2 | 2.96 |
| S. agalactiae | | 2.16 | 2.68 | 3.44 |
| B19 | | | | 3.81 |
| VZV | | | | 2.59 |

1. A method of identifying genomic regions that are differentially methylated in two samples, the method comprising:
a) providing two samples comprising DNA;
b) contacting the samples with one or more CpG methylation-sensitive restriction enzyme to generate cut sites or nick sites in the DNA at enzyme recognition sites;
c) ligating adapters to the cut sites or nick sites to generate DNA libraries;
d) sequencing the DNA libraries to generate sequencing reads;
e) mapping the sequencing reads to a reference genome; and
f) comparing the mapped sequencing reads from each sample to identify genomic regions that are differentially methylated in the two samples, wherein the method further comprises terminally dephosphorylating the DNA prior to step (b).

2. (canceled)

3. The method of claim 1, wherein the activity of the one or more CpG methylation-sensitive restriction enzyme is blocked by CpG methylation within or adjacent to its cognate recognition site.

4. The method of claim 3, wherein the one or more CpG methylation-sensitive restriction enzyme comprises a restriction enzyme selected from the group consisting of AatII, AccII, AluI, Aor13HI, Aor51HI, BspT104I, BssHII, Cfr10I, ClaI, CpoI, DdeI, Eco52I, HaeII, HapII, HhaI, HpyCH4IV, HpaII, HaeIII, MluI, NaeI, NotI, NruI, NsbI, Nt.CviPII, PmaCI, Psp1406I, PvuI, RsaI, SacII, SalI, SmaI, SnaBI, and Sau3AI.

5. The method of claim 4, wherein the one or more CpG methylation-sensitive restriction enzyme comprises at least one of AluI, DdeI, HpyCH4IV, HpaII, HaeIII, RsaI, and Sau3AI.

6. The method of claim 5, wherein the one or more CpG methylation-sensitive restriction enzyme comprises at least four of AluI, DdeI, HpyCH4IV, HpaII, HaeIII, RsaI, and Sau3AI.

7. The method of claim 6, wherein the one or more CpG methylation-sensitive restriction enzyme comprises all seven of AluI, DdeI, HpyCH4IV, HpaII, HaeIII, RsaI, and Sau3AI.

8. The method of claim 4, wherein the one or more CpG methylation-sensitive restriction enzyme comprises Nt.CviPII.

9. The method of claim 1, wherein the activity of the one or more CpG methylation-sensitive restriction enzyme requires CpG methylation within or adjacent to its cognate recognition site.

10. The method of claim 9, wherein the one or more CpG methylation-sensitive restriction enzyme comprises a restriction enzyme selected from the group consisting of AbaSI, FspEI, LpnPI, MspJI, and McrBC.

11. The method of claim 10, wherein the one or more CpG methylation-sensitive restriction enzyme comprises FspEI and MspJI.

12. The method of claim 1, wherein the adapters are ligated to the cut/nick sites but not to uncut/unnicked sites in step (c).

13. A composition comprising at least four CpG methylation-sensitive restriction enzymes selected from the group consisting of AluI, DdeI, HpyCH4IV, HpaII, HaeIII, RsaI, and Sau3AI.

14. (canceled)

15. A method of generating a DNA library that is depleted of CpG-methylated DNA, the method comprising:
a) providing a sample comprising DNA;
b) contacting the sample with the composition of claim 13 or NtCviPII to generate cut sites in the DNA at enzyme recognition sites that lack CpG methylation; and
c) ligating adapters to the cut sites to generate a DNA library that is depleted of CpG-methylated DNA.

16. (canceled)

17. A method of generating a DNA library that is enriched for CpG-methylated DNA, the method comprising:
a) providing a sample comprising DNA;
b) contacting the sample with FspE1 and MspJ1 to generate cut sites in the DNA at FspE1 and MspJ1 recognition sites comprising CpG methylation; and
c) ligating adapters to the cut sites to generate a DNA library that is enriched for CpG-methylated DNA.

18. The method of claim 15 further comprising terminally dephosphorylating the DNA prior to step (b).

19. The method of claim 15, wherein the sample comprises DNA from a mammalian organism and DNA from a pathogenic organism.

20. The method of claim 19, wherein the mammalian organism is a human.

21. The method of claim 19, wherein the pathogenic organism is a bacterium, a yeast, or a virus.

22. The method of claim 15, further comprising an additional depletion and/or enrichment step.

23. The method of claim 22 wherein the additional depletion and/or enrichment step comprises: contacting the sample after step (c) with a nucleic acid-guided nuclease and guide nucleic acids (gNAs), wherein the gNAs are complementary to sites within DNA molecules that are targeted for depletion, thereby generating cut DNA molecules that are adapter-ligated on only one end.

24. (canceled)
